(19) United States
(12) Patent Application Publication
(10) Pub. No.: US 2026/0106020 A1
(43) Pub. Date: Apr. 16, 2026

(54) SYSTEM AND METHOD FOR CELL-OF-ORIGIN CLASSIFICATION BASED ON INTERPRETABLE CELLULAR FEATURES

(71) Applicants: Genentech, Inc., South San Francisco, CA (US); Ventana Medical Systems, Inc., Tucson, AZ (US)

(72) Inventors: Qiangqiang GU, Tucson, AZ (US); Nazim SHAIKH, Tucson, AZ (US); Ping-chang LIN, Tucson, AZ (US); Srinath JAYACHANDRAN, Tucson, AZ (US); Prasanna PORWAL, Tucson, AZ (US); Xiao LI, Tucson, AZ (US); Yao NIE, Tucson, AZ (US)

(21) Appl. No.: 19/422,305

(22) Filed: Dec. 16, 2025

Related U.S. Application Data
(63) Continuation of application No. PCT/US2024/036447, filed on Jul. 1, 2024.

(30) Foreign Application Priority Data
Jun. 30, 2023 (IN) 202311044011

(51) Int. Cl. G16H 30/40

(52) U.S. Cl. CPC G16H 30/40

(57) ABSTRACT

A method of classifying a tissue sample by a classification system includes identifying, by the classification system, a plurality of tiles corresponding to whole-slide image data of the tissue sample; generating, by the classification system, a plurality of semantic masks corresponding to the plurality of tiles, each one of the plurality of semantic masks identifying a cell boundary and a cell type of each cell within a corresponding tile of the plurality of tiles; generating, by the classification system, a plurality of cellular features for each tile of the plurality of tiles based on a corresponding one of the plurality of semantic masks; and classifying, by the classification system, the tissue sample based on the plurality of cellular features for each one of the plurality of tiles.

### CROSS-REFERENCE TO RELATED APPLICATION(S)

This application is a bypass continuation application of International Application No. PCT/US2024/036447, filed Jul. 1, 2024, which claims priority to, and the benefit of, Indian Application No. 202311044011 (“INTERPRETABLE FEATURE BASED NETWORK FOR CLASSIFYING CELL-OF-ORIGIN FROM WHOLE SLIDE IMAGES IN DIFFUSE LARGE B-CELL LYMPHOMA PATIENTS”), filed on Jun. 30, 2023, with the Indian Patent Office, the entire content of which is incorporated herein by reference.

### FIELD

Aspects of some embodiments of the present disclosure relate to a system and method for tissue sample classification.

### BACKGROUND

Cancers in their various forms have become one of the leading causes of death worldwide. Diffuse large B-cell lymphoma (DLBCL), which accounts for about 25% to 30% of all the non-Hodgkin lymphomas, is an aggressive and the most common type of lymphoma. Although about two-thirds of DLBCL patients can be cured with standard treatment, research has focused on determining which patients have less favorable prognosis so that they can be considered for novel targeted-treatment strategies. Germinal center B-cell-like (GCB) and activated B-cell-like (ABC) are two major biologically distinct molecular subtypes of DLBCL. Patients with the ABC DLBCL generally have worse prognosis than the GCB DLBCL patients when treated with combined therapy R-CHOP (i.e., a combination of chemotherapy and targeted therapy drugs used to treat cancer). Therefore, cell-of-origin (COO) classification or its surrogates have been incorporated into the clinical practice and clinical trials to help better understand DLBCL biological heterogeneity and enable researchers to develop more accurate therapeutic targeting strategies.

Well-established COO classification algorithm uses gene expression profiling (GEP). However, as GEP is not widely accessible, researchers and pathologists in clinical practice approximate molecular subtypes using immunohistochemical (IHC) patterns such as the most widely used Hans algorithm, where expert visual assessment of multiple IHC assays are required. Due to the imperfection of IHC in assessing molecular subtype, more precise strategies are under development.

The above information disclosed in this Background section is only for enhancement of understanding of the background and therefore the information discussed in this Background section does not necessarily constitute prior art.

### SUMMARY

Aspects of some embodiments of the present disclosure are directed to a system and method for standardized and automated cell-of-origin (COO) classification based on hematoxylin and eosin (H&E) stained whole-slide-images (WSIs), which are readily available from primary diagnosis and thus tissue-saving and potentially more efficient by shortening the turnaround time. In some embodiments, the classification system leverages both interpretable cellular features derived from image tiles and an attention based multi-instance learning (AMIL) framework to provide classifications based on a single WSI of a tissue sample.

According to some embodiments, the classification system first performs nuclei segmentation and classification to identify each nucleus in each tile of a WSI and to classify them into different phenotypes. Then, the classification system derives interpretable cellular features from nuclei in each image tile and uses them to generate a tile-level histopathological representation for the image tile. Lastly, the classification system utilizes an attention based multi-instance learning (AMIL) framework to aggregate all tile-level histopathological representations from a WSI to form the slide-level representation and to classify the whole slide image.

According to some embodiments of the present disclosure, there is provided a method of classifying a tissue sample by a classification system based on machine learning, the method including: identifying, by the classification system, a plurality of tiles corresponding to whole-slide image data of the tissue sample; generating, by the classification system, a plurality of semantic masks corresponding to the plurality of tiles, each one of the plurality of semantic masks identifying a cell boundary and a cell type of each cell within a corresponding tile of the plurality of tiles; generating, by the classification system, a plurality of cellular features for each tile of the plurality of tiles based on a corresponding one of the plurality of semantic masks; and classifying, by the classification system, the tissue sample based on the plurality of cellular features for each one of the plurality of tiles.

In some embodiments, the identifying the plurality of tiles includes: receiving, by the classification system, the whole-slide image data corresponding to the tissue sample; and extracting, by the classification system, the plurality of tiles from the whole-slide image data.

In some embodiments, the whole-slide image data includes at least one digitized image of the tissue sample of a patient that is stained with hematoxylin and eosin (H&E) dyes or a region-of-interest (ROI) map.

In some embodiments, the identifying the plurality of tiles further includes: performing stain normalizing, by the classification system, based on the plurality of tiles to generate a plurality of normalized tiles.

In some embodiments, the performing stain normalizing includes: generating, by a first model of the classification system, the plurality of normalized tiles based on the plurality of tiles, wherein the first model includes a fully convolutional neural network.

In some embodiments, the generating the semantic masks includes: encoding, by an encoder of the classification system, the a tile of the plurality of tiles to generate encoded data corresponding to the tile; generating, by a segmentation decoder of the classification system, a segmentation mask corresponding to the tile based on the encoded data, the segmentation mask identifying the cell boundary of each cell within the tile; and classifying, by a classification decoder of the classification system, the cell type of each cell within the segmentation mask as one of a plurality of cell type categories; generating a semantic mask of the plurality of semantic masks to indicate the cell boundary and the cell type of each cell with the tile.

In some embodiments, the plurality of cell type categories includes a tumor cell, a lymphocyte cell, and other.

In some embodiments, the generating the plurality of cellular features for each tile of the plurality of tiles includes: generating a plurality of nuclear-level features for the tile based on the corresponding one of the plurality of semantic masks; generating a plurality of tile-level features by aggregating the plurality of nuclear-level features; and extracting the plurality of cellular features from the plurality of tile-level features.

In some embodiments, the generating the plurality of nuclear-level features for the tile includes: computing a plurality of nuclear morphology features for each cell having a tumor cell type within the corresponding one of the plurality of semantic masks, wherein the plurality of nuclear morphology features includes at least one of: basic geometric features including shape, size, and circularity of a nucleus of the cell having the tumor cell type; first-order statistics of gray-level intensity inside the nucleus; texture features derived from gray-level co-occurrence matrix of the nucleus; advanced morphology features for characterizing irregularity of the nucleus; chromatin distribution features of the nucleus; nuclear boundary signature of the nucleus; or curvature features of the nucleus, and wherein the plurality of nuclear-level features includes a collection of nuclear morphology features of all nuclei of cells having the tumor cell type within the corresponding one of the plurality of semantic masks.

In some embodiments, the generating the plurality of tile-level features includes: calculating a statistical mean of ones of the nuclear-level features associated with cells having a tumor cell type within the corresponding one of the plurality of semantic masks to generate a mean vector; calculating a standard deviation of the ones of the nuclear-level features associated with cells having the tumor cell type to generate a standard deviation vector; and determining spatial distribution features of cells within the corresponding one of the plurality of semantic masks to generate one or more spatial distribution vectors, wherein the plurality of tile-level features includes the mean vector, the standard deviation vector, and the one or more spatial distribution vectors.

In some embodiments, the spatial distribution features includes: density of each type of cell of cells within the corresponding one of the plurality of semantic masks; and average distances between cells within the corresponding one of the plurality of semantic masks.

In some embodiments, the extracting the plurality of cellular features includes: removing one or more of the plurality of tile-level features that have low variance across cells or high correlation with other ones of the plurality of tile-level features; and normalizing remaining ones of the tile-level features to generate the plurality of cellular features.

In some embodiments, the classifying the tissue sample includes: generating, by an attention-based aggregator of the classification system, slide-level features based on the plurality of cellular features for each one of the plurality of tiles; and classifying, by a slide classifier of the classification system, the tissue sample based on the slide-level features.

In some embodiments, the classifying of the tissue sample includes: identifying the tissue sample as containing a first subtype of diffuse large B-cell lymphoma (DLBCL) or a second subtype of DLBCL.

In some embodiments, the first subtype includes a germinal center B-cell-like (GCB) subtype, and the second subtype includes an activated B-cell-like (ABC) subtype.

According to some embodiments of the present disclosure, there is provided a classification system for classifying a tissue sample, the classification system including: a processor; and a memory storing instructions that, when executed on the processor, cause the processor to perform: identifying a plurality of tiles corresponding to whole-slide image data of the tissue sample; generating a plurality of semantic masks corresponding to the plurality of tiles, each one of the plurality of semantic masks identifying a cell boundary and a cell type of each cell within the tile; generating a plurality of cellular features for each tile of the plurality of tiles based on a corresponding one of the plurality of semantic masks; and classifying the tissue sample based on the plurality of cellular features for each one of the plurality of tiles.

In some embodiments, the generating the semantic masks includes: encoding, by an encoder of the classification system, the a tile of the plurality of tiles to generate encoded data corresponding to the tile; generating, by a segmentation decoder of the classification system, a segmentation mask corresponding to the tile based on the encoded data, the segmentation mask identifying the cell boundary of each cell within the tile; and classifying, by a classification decoder of the classification system, the cell type of each cell within the segmentation mask as one of a plurality of cell type categories; generating a semantic mask of the plurality of semantic masks to indicate the cell boundary and the cell type of each cell with the tile, and wherein the plurality of cell type categories includes a tumor cell, a lymphocyte cell, and other.

In some embodiments, the generating the plurality of cellular features for each tile of the plurality of tiles includes: generating a plurality of nuclear-level features for the tile based on the corresponding one of the plurality of semantic masks; generating a plurality of tile-level features by aggregating the plurality of nuclear-level features; and extracting the plurality of cellular features from the plurality of tile-level features.

In some embodiments, the generating the plurality of tile-level features includes: calculating a statistical mean of ones of the nuclear-level features associated with cells having a tumor cell type within the corresponding one of the plurality of semantic masks to generate a mean vector; calculating a standard deviation of the ones of the nuclear-level features associated with cells having the tumor cell type to generate a standard deviation vector; and determining spatial distribution features of cells within the corresponding one of the plurality of semantic masks to generate one or more spatial distribution vectors, wherein the plurality of tile-level features includes the mean vector, the standard deviation vector, and the one or more spatial distribution vectors.

In some embodiments, the classifying the tissue sample includes: generating, by an attention-based aggregator of the classification system, slide-level features based on the plurality of cellular features for each one of the plurality of tiles; and classifying, by a slide classifier of the classification system, the tissue sample based on the slide-level features.

### BRIEF DESCRIPTION OF THE DRAWINGS

Non-limiting and non-exhaustive embodiments according to the present disclosure are described with reference to the following figures, wherein like reference numerals refer to like parts throughout the various views unless otherwise specified.

FIG. 1 is a block diagram illustrating the classification system, according to some embodiments of the present disclosure.

FIG. 2 is a block diagram illustrating a whole slide image preprocessor and its operation, according to some embodiments of the present disclosure.

FIG. 3 is a block diagram illustrating the internal structure of a nuclei segmentation and classification block, according to some embodiments of the present disclosure.

FIG. 4 is a block diagram illustrating a cell feature extractor, according to some embodiments of the present disclosure.

FIG. 5 is a block diagram illustrating an attention based multi-instance learning (AMIL) model, according to some embodiments of the present disclosure.

FIG. 6 is a flow diagram illustrating a process of classifying a tissue sample by the classification system, according to some embodiments of the present disclosure.

### DETAILED DESCRIPTION

Hereinafter, aspects of some example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

Aspects of the embodiments of the present disclosure are directed to a system for standardized and automated cell-of-origin (COO) prediction based on hematoxylin and eosin (H&E) stained whole-slide-images (WSIs), which are readily available from primary diagnosis.

In the related art, attempts have been made to utilize deep learning approaches for COO prediction of patients with diffuse large B-cell lymphoma (DLBCL) using H&E WSIs; however, no insights may be derived about the relation between histopathology features and different molecular subtypes.

According to some embodiments, a classification system utilizing a cellular feature based interpretable network to perform nuclei segmentation and classification to identify each nucleus in each image tile of a WSI and to classify them into different phenotypes. The classification system then derives interpretable cellular features from nuclei in each image tile and uses them as the tile-level histopathological representation. Further, the classification system also utilizes an attention-based multi-instance learning (AMIL) framework to aggregate all tile-level histopathological representations from a WSI to form the slide-level representation, and to classify the slide based on the slide-level representation. The comprehensive, quantifiable, and generic cellular feature set generated by the classification system may characterize nuclei morphologies, spatial patterns, as well as phenotype compositions, which allows the feature set to be used to train diverse machine learning models for clinical predictions. In some examples, the weakly-supervised AMIL model may classify the germinal center B-cell-like (GCB) and activated B-cell-like (ABC) molecular subtypes of DLBCL with superior performance as compared to approaches of the related art. Also, when compared to the related art, the use of the classification system according to the present disclosure is favorable in terms of explainability, as the model behavior can be interpreted through both attention scores and biologically relevant feature importances at whole slide as well as image tile levels.

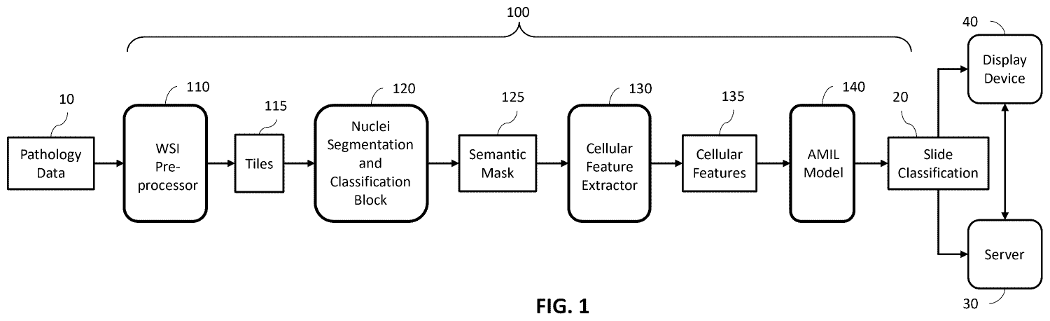

FIG. 1 is a block diagram illustrating the classification system 100, according to some embodiments of the present disclosure.

According to some embodiments, the classification system (e.g., the cell-of-origin classification system) 100 is configured to analyze a whole slide image (WSI) of a tumorous tissue sample to extract biologically relevant features at the whole slide as well as image tile levels, to apply attention scores to the extracted features, and to then classify the molecular subtype of the tissue sample. In some embodiments, the classification system 100 utilizes machine learning-based models to perform cell-of-origin (COO) classification. In some examples, the classification system may classify the molecular subtypes of diffuse large B-cell lymphoma (DLBCL) contained in the tissue sample as either germinal center B-cell-like (GCB) or activated B-cell-like (ABC) subtypes. However, embodiments of the present disclosure are not limited thereto, and the classification system 100 may be utilized to classify any suitable molecular subtype of any suitable type of cancer. The classification system 100 may also classify a tissue sample as “unknown”.

The WSI data 102 that is supplied to the classification system 100 may include one or more digitized images of a tissue sample (e.g., a tumorous tissue sample) of the patient that is stained with hematoxylin and eosin dyes. H&E dyes stain cell nuclei, extracellular matrix and cytoplasm, and other cell structures, with different colors thus allowing a pathologist and the classification system 100 to differentiate between different cellular structure. Also, the overall patterns of coloration from the stain show the general layout and distribution of cells and provide a view of a tissue sample's structure. In some examples, the whole-slide image data 102 may include one or more image tiles that are extracted from (e.g., randomly selected and extracted from) a viable tumor region of a stained tissue sample.

The slide classification/prediction 20 that is output by the classification system 100 may be a multi-level output (e.g., ‘0’, ‘1’, ‘2’, etc.) indicating the classification category for which classification system 100 is trained. In some examples, slide classification 20 may be a confidence level or probability associated with the various classification categories. However, these are merely examples, and embodiments of the present disclosure are not limited thereto.

According to some embodiments, the classification system 100 includes a segmentation and classification block 120, a cellular feature extractor 130, and an attention based multi-instance learning (AMIL) model 140.

In some embodiments, the segmentation and classification block 120 is configured to is configured to receive a plurality of tiles 115 corresponding to WSI data 10 of a tissue sample, to analyze the tiles 115 at the tile level, and to generate a plurality of semantic masks 125 corresponding to the plurality of tiles 115, where each one of the plurality of semantic masks 125 identifies a cell boundary and a cell type of each cell within the tiles 115. The cellular feature extractor 130 is configured to generate a plurality of cellular features 135 for each tile 115 based on a corresponding one of the semantic masks 125. The AMIL model 140, in turn, classifies the tissue sample based on the cellular features 135 for each one of the tiles 115.

Once the classification system 100 outputs a slide classification/prediction (which may also be referred to as a COO prediction) 20, the output may be transmitted to a server (e.g., a remote server or a cloud server) 30 for further processing and/or to a display device 40 for display to a user.

In some embodiments, the classification system 100 also includes a WSI processor 110 that is configured to preprocess the WSI data 10 to ensure uniformity in the tiles that are supplied to the segmentation and classification block 120. Given that different labs that generate whole slide images based on tissue samples may use different stainers and/or settings, the resulting WSIs produced by such labs may have different stains (e.g., different colorations). Therefore, in some embodiments, the WSI processor 110 performs stain normalization, that is, standardizes the stains across all tiles, and generates a plurality of normalized tiles that are then passed onto the segmentation and classification block 120 for further analysis and processing. The WSI processor 110 may also perform the function of extracting tiles from an original WSI.

However, embodiments of the present disclosure are not limited thereto. For example, one or more functions of the WSI processor 110 may be omitted from this component and integrated into other component blocks, or omitted from the classification system 100 altogether. In some examples, stain normalization may be omitted from the WSI processor 110 and the function may be integrated into the input stage of segmentation and classification block 120. Further, the stain normalization function may be omitted from the classification system 100 and the segmentation and classification block 120 may operate on the raw tiles with potentially different staining profiles.

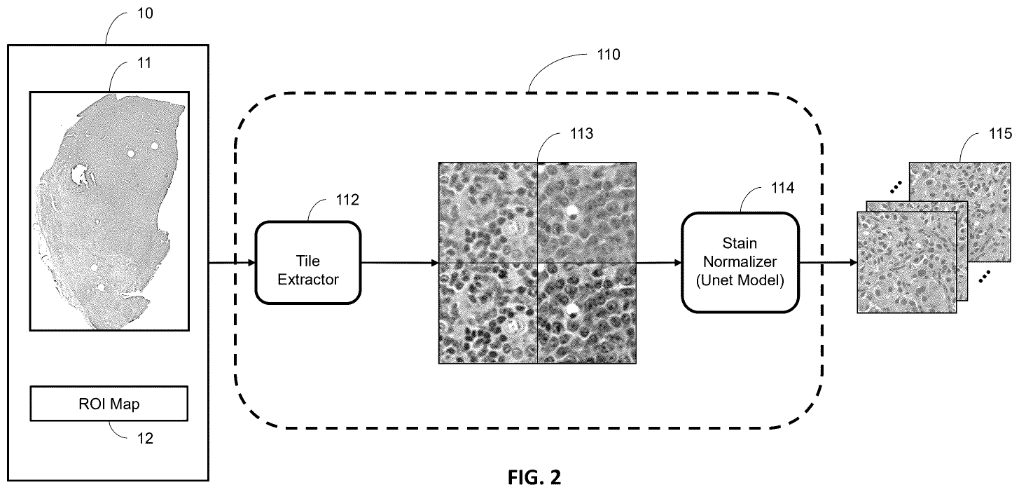

FIG. 2 is a block diagram illustrating the WSI processor 110 and its operation, according to some embodiments of the present disclosure.

In some examples, the WSI data 10 includes a WSI 11 and a region-of-interest (ROI) map 12 that identifies the potential tumor regions of the WSI 11 that are relevant to the analysis of the classification system 100. The ROI map 12 may be manually annotated by a pathologist.

In some examples, the ROI map 12 may be generated by applying a series of filters to the WSI 11. The filters may include at least one of a background filter, an out-of-focus filter, a crush filter, a pen mark filter, a hemorrhage filter, a necrosis filter, a fat tissue filter, or non-lymphoid filter. The background filter may remove portions of the WSI 11 that do not contain any tissue by detecting portions that contain tissue and discarding everything else. The out-of-focus filter may remove portions of the WSI 11, which contain tissue that are not in focus, i.e., blurry either due to suboptimal image acquisition or slide preparation. The crush filter may remove portions of the WSI 11 that contain tissue with crush artifacts, i.e., clusters of cells that were deformed or damaged due to suboptimal tissue handling. The pen mark filter may remove portions of the WSI 11 that contain pen marks made on the physical glass slide as may be common in anatomical pathology labs. The hemorrhage filter may remove portions of the WSI 11 that contain tissue with signs of bleeding, i.e., excessive extravascular accumulation of red blood cells that obscures tumor tissue. The necrosis filter may remove portions of the WSI 11 that contain necrotic tissue. This can demonstrate a range of features from eosinophilic tissue debris without intact tumor cells to cells with nuclear changes including pyknosis, karyorrhexis, karyolysis and cytoplasmic vacuolization and/or eosinophilia. The fat tissue filter may remove portions of the WSI 11 that contain fat tissue including adipocytes and associated connective tissue. The non-lymphoid tissue filter may remove portions of the WSI 11 that contain lymphoid tissue. The filter may identify lymphoid tissues, which may be encountered at anatomic sites at which lymphomas can occur, e.g., lymphoma tissue, lymph node parenchyma, lymphoid-rich stroma, lymphoid aggregates in nodal and extranodal anatomic sites. Positively classified WSI portions containing lymphoid tissue may be kept and negatively classified areas not containing lymphoid tissue may be removed.

Each of said filters may represents a function parametrized by a convolutional neural network (CNN) that takes a WSI or a portions thereof as input and returns a single Boolean value as output. The CNN model underlying each filter may be trained to identify a specific histologic concept in WSIs and to classify its portions depending on whether the concept is present or absent. An output of zero may mean that the component did not identify the concept in a given WSI portion, and an output of one may means that the filter did identify the concept in the WSI portion.

The application of the above noted filters produces an analysis region of interest as output. This region that may be continuous or may be spread out in multiple parts, i.e., not continuously connected. In some examples, the region identified by the ROI map 12 includes areas enriched with lymphoid elements (e.g., lymphoma tissue, lymph node parenchyma, lymphoid-rich stroma, lymphoid aggregates) that may be encountered in nodal and extranodal anatomic sites. The ROI may be free of artifacts and non-lymphoid tissue (e.g., be free of background regions, out-of-focus regions, crush tissue, pen mark regions, hemorrhage tissue, necrotic tissue, and fat tissue). In some examples, the ROI map 12 may be further examined and modified by a human user (e.g., pathologist) as desired.

In some embodiments, the WSI processor 110 includes a tile extractor 112 and a stain normalizer 114.

The tile extractor 112 may apply the ROI map 12 to (e.g., overlay the ROI map 12 onto) the WSI 11 to identify regions of interest in the WSI 11 and to then extract a plurality of non-overlapping tiles 113 of equal size from the regions of interest in the WSI 11. In some examples, the tile extractor 112 may also extract tiles from the WSI 11 and discard those tiles that do not fall within the ROI (e.g., tiles that have greater than 10% overlap with non-ROI regions). In some examples, the tile extractor 112 may extract a number of (e.g., more than 10,000) non-overlapping tiles of 1024×1024 pixels from the WSI 11, which may have been digitized at 40× magnification. The 1024×1024 pixel image size may ensure that each tile contains a sufficient number of cells to derive robust cellular feature statistics.

To accommodate for the different stains that the tiles 113 may exhibit (as, e.g., represented by tiles 113a, 113b, 113c, and 133d), the stain normalizer 114 standardizes the stains across the plurality of tiles to generate a plurality of normalized tiles 115 that that have a uniform stain irrespective of the stain used in the WSI 11.

In some embodiments, the stain normalizer 114 includes a first model, which may utilize a U-Net architecture having a neural network (e.g., a convolutional neural network) that expresses an input image in short form as a vector and then upscales the image in the desired (e.g., standardized) stain. However, embodiments of the present disclosure are not limited thereto, and the first model of the stain normalizer 114 may use any suitable architecture.

The stain normalizer 114 provides the normalized tiles 115 to the nuclei segmentation and classification block 120 to generate semantic masks.

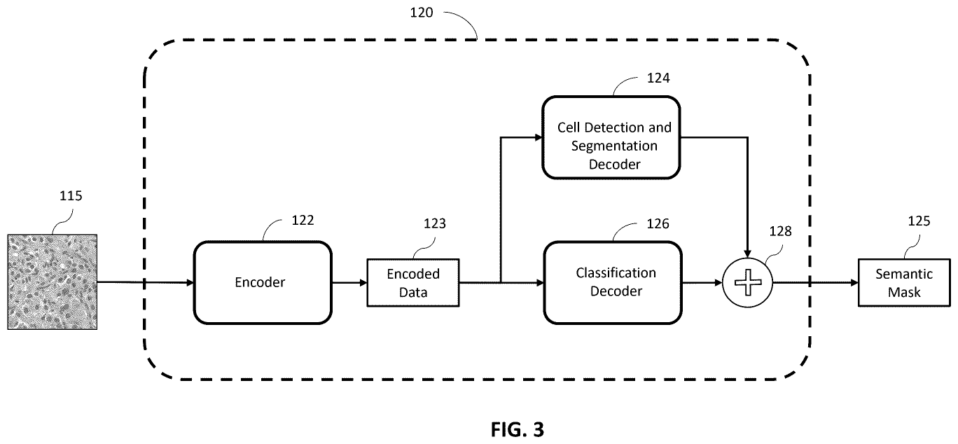

FIG. 3 is a block diagram illustrating the internal structure of the nuclei segmentation and classification block 120, according to some embodiments of the present disclosure.

Referring to FIG. 3A, in some embodiments, the segmentation and classification block 120 includes an encoder 122, a segmentation decoder (e.g., a cell detection and segmentation decoder) 124, a classification decoder 126, and an aggregator 128.

The encoder 122 receives the plurality of tiles (e.g., non-overlapping normalized tiles) 115 and encodes each of the tiles 115 to generate encoded data 123 corresponding to each of the tiles 115. The encoder 122 may reduce the dimensionality of the information from the input tile 115 (e.g., a 1024×1024 pixel image) to a single embedding vector (e.g., a vector of length 1024) to make it easier to process by the decoders 124 and 126. In some examples, the encoder 122 may extract relevant information from the tile image 115 by performing a series of non-sampling or downsampling/pooling of certain sections of the tile 115. In some examples, the encoder 122 may have a residual network (ResNet)-based architecture (e.g., ResNet15/50).

The segmentation decoder 124 is configured to detect cells (e.g., the cell nuclei) within a tile 115 and to generate a segmentation mask that defines the contours of each cell and effectively separates each cell (e.g., nuclei) from the background. The segmentation decoder 124 may be a deep learning based neural network trained for object detection, such as a StarDist network that is trained to distinguish between cells (e.g., nuclei) and background.

The classification decoder 126 classifies the cell type of each cell within the tile 115 as one of a plurality of cell type categories (or phenotypes) including a “tumor cell”, a “lymphocyte cell”, and “other cell”. In some embodiments, the classification decoder 126 includes a neural network, such as a convolutional neural network (ConvNet/CNN), a recurrent neural network (RNN), or the like, which is capable of cell classification.

The aggregator 128 aggregates the outputs of the decoders 124 and 126 to generate a semantic mask 125 that indicates the cell boundary and the cell type of each cell with the tile 115.

While the segmentation decoder 124 and the classification decoder 126 are shown in FIG. 3 as operating in parallel on the encoded data 123, embodiments of the present disclosure are not limited thereto, and the two decoders 124 and 126 may operate in series. For example, the classification decoder 126 may receive the segmentation mask as input and generate the semantic mask 125 as output (thus obviating the need for the aggregator 128).

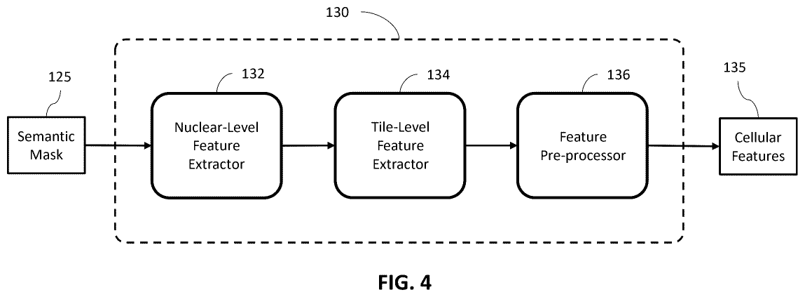

FIG. 4 is a block diagram illustrating the cellular feature extractor 130, according to some embodiments of the present disclosure.

Referring to FIG. 4, in some embodiments, the cellular feature extractor 130 includes a nuclear-level feature extractor (or cell-level feature extractor) 132, a tile-level feature extractor 134, and a feature pre-processor 136.

The nuclear-level feature extractor 132 is configured to generate a plurality of nuclear-level features for each tile 115 based on the corresponding one of the semantic masks 125. In some embodiments, the nuclear-level feature extractor 132 receives a semantic mask 125 and computes a plurality of nuclear morphology features for each cell having a tumor cell type within the semantic mask 125.

In some examples, the nuclear morphology features may include: 1) basic geometric features, such as shape, size, and circularity of a nucleus of the cell having the tumor cell type; 2) first-order statistics of gray-level intensity inside the nucleus; 3) texture features derived from gray-level co-occurrence matrix of the nucleus; 4) advanced morphology features for characterizing irregularity of the nucleus; and/or 5) chromatin distribution features of the nucleus; nuclear boundary signature and curvature features of the nucleus. However, embodiments of the present disclosure are not limited thereto, and the computed the nuclear morphology features may include any other suitable features that may be relevant to the classification performed by the classification system 100. Here, the nuclear-level features include the collection/totality of nuclear morphology features of all nuclei of cells having the tumor cell type within the received semantic mask 125.

In some examples, the nuclear-level feature extractor 132 may extract 210 features for each cell within the semantic mask 125.

The tile-level feature extractor 134 is configured to generate a plurality of tile-level features by aggregating the plurality of nuclear-level features. In some embodiments, the tile-level feature extractor 134 is configured to calculate a statistical mean of ones of the nuclear-level features associated with cells having a tumor cell type within the corresponding the semantic mask 125 to generate a mean vector (of, e.g., length 210), and to calculate a standard deviation of the same ones of the nuclear-level features to generate a standard deviation vector (of, e.g., length 210).

In some embodiments, the tile-level feature extractor 134 is configured to determine spatial distribution features of cells within the semantic mask 125, such as density of each cell phenotype and average distances between cells, to generate one or more spatial distribution vectors. The spatial patterns may be captured through graph-based methods such as k-nearest neighbor (KNN) graphs, or the like.

The tile-level features output by the tile-level feature extractor 134 may include the mean vector, the standard deviation vector, and the one or more spatial distribution vectors.

In some examples, the cellular feature extraction may be performed entirely based on image processing and computer vision and may not utilize a neural network.

According to some embodiments, the feature pre-processor 136 is configured to extract the plurality of cellular features from the plurality of tile-level features. Due to general similarity between each nucleus, some cellular features may have low variance across cells or high correlations with other cellular features. As such, the feature pre-processor 136 may exclude these features by applying predefined variance and correlation thresholds. In some examples, this may result in a 336-dimensional feature representation for each tile 115. Finally, the feature pre-processor 136 applies normalization on the remaining (i.e., non-excluded) tile-level features to generate the plurality of tile-level cellular features, which are output to the AMIL model 140.

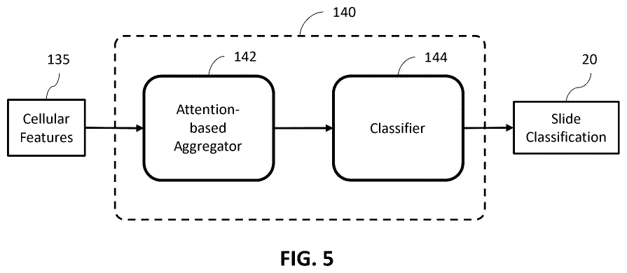

FIG. 5 is a block diagram illustrating the AMIL model 140, according to some embodiments of the present disclosure.

In some embodiments, the AMIL model 140 includes an attention-based aggregator 142 and a slide classifier 144. The attention-based aggregator 142 is configured to receive the plurality of cellular features corresponding to all of the tiles 115 within the WSI 10 and to generate slide-level features based on all of cellular features, and the slide classifier 144 is configured to classifying the tissue sample/WSI 10 based on the slide-level features. In so doing, according to some examples, the slide classifier 144 may identify the tissue sample/WSI 10 as containing a first subtype of diffuse large B-cell lymphoma (DLBCL), such as a germinal center B-cell-like (GCB) subtype, or as containing a second subtype of DLBCL, such as an activated B-cell-like (ABC) subtype.

The attention-based aggregator 142 may utilize softmax attention to highlight the contributions of the individual tile-level cellular features to the whole, such that different instances' (i.e., tile-level cellular features') contributions are no longer considered equally and are weighed differently. For example, the attention-based aggregator 142 may prioritizes dense tumor tiles while assigning lesser importance to tiles 115 depicting open spaces or non-tumor tissue. This inherently reduces the impact of noisy samples and improves the prediction performance. In some examples, the attention-based aggregator 142 may include multiple linear layers, each followed by a dropout, 1-dimension batchnorm and Relu activation.

The slide classifier 144 includes a multi-instance learning (MIL) classifier that may include multilayer perceptron (MLP) with dropout and Relu activation, in some examples.

The classification system 100 represents a deep learning network that is interpretable due the use of cellular features that are quantifiable and derived from known biological features, and because the performance of the network can be attributed to some of these features.

The classification system 100 offers a dual interpretability approach utilizing attention scores and SHAP (SHapley Additive exPlanations) values and visualizations (which aid in explaining the output of the machine learning system). For example, attention heatmaps based on the output of the attention-based aggregator 142 may be used to visually indicate the portions of a WSI that the classification system 100 deems more important (e.g., dense tumor tiles). Further, SHAP summary plots may be used to show the impact on the slide classification/prediction of the most important features (based on the output of the attention-based aggregator 142). For example, it may be revealed that tumor density is the most important feature, and higher tumor density value may points towards a prediction of “ABC” subtype. Meanwhile, higher variation of the nuclear texture may also be shown to be associated with the “ABC” subtype. Knowing that ABC DLBCL patients generally have worse prognosis than the GCB DLBCL patients, this observation can be linked to the report that within the morphologic spectrum of DLBCL, certain cases of aggressive mature B-cell non-hodgkin lymphoma have some of the morphologic features of burkitt lymphoma but have greater nuclear and cytoplasmic variability.

Additionally, to gain insights about model behavior on a local region, the slide classifier 144 can be applied to a tile-level representation and use SHAP waterfall plot to display individual tile prediction explanations. For example, SHAP waterfall plots may be used to visualize the contribution of the important features in deriving the slide classification/prediction 20. As an example, such visualizations may reveal that the shape and boundary features are the main determinants for predicting the tile as GCB, while morphological variations are the dominating factors for a ABC prediction.

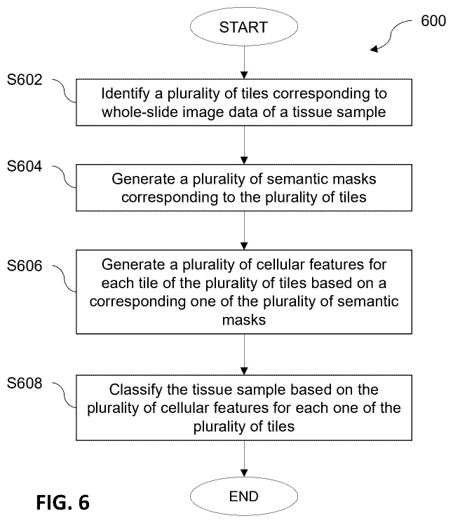

FIG. 6 is a flow diagram illustrating a process 600 of classifying a tissue sample by the classification system 100, according to some embodiments of the present disclosure.

In some embodiments, the classification system 100 identifies a plurality of tiles 115 corresponding to whole-slide image data 10 of the tissue sample (S602).

The classification system 100 generates a plurality of semantic masks 125 corresponding to the plurality of tiles 115 (S604). Each one of the plurality of semantic masks 125 identifies a cell boundary and a cell type of each cell within the tile 115.

The classification system 100 generates a plurality of tile-level cellular features 135 for each tile 115 based on a corresponding one of the plurality of semantic masks 125 (S606).

The classification system 100 then classifies the tissue sample based on the plurality of tile-level cellular features 135 for each one of the plurality of tiles 115 (S608).

Accordingly, as described above, the classification system 100 elegantly combines quantified and interpretable tile-level cellular features and an AMIL network architecture in an effective way to generate a slide-level classification/prediction 20 pertaining to a tissue sample. The notable performance in COO prediction for DLBCL patients demonstrates the descriptive power of the designed cellular features, as well as the effectiveness of attention mechanisms in weighing the relevance of tissue image tiles to the target prediction. The combination also greatly enhances model interpretability through both attention scoring and SHAP analysis, each of which is only applicable to either pure deep neural network based approaches or conventional machine learning approaches alone. As a result, the classification system 100 allows a holistic interpretation and analysis of model behavior at various levels, covering the entire dataset, whole slide images, and individual image tiles. Moreover, the linkage to each individual cellular feature can be established at all levels, enabling more model transparency and trustworthiness and making the classification system 100 a great biomarker discovery tool for clinical predictions.

According to various embodiments of the present disclosure, the classification system 100 is implemented using one or more processing circuits or electronic circuits configured to perform various operations as described above. Types of electronic circuits may include a central processing unit (CPU), a graphics processing unit (GPU), an artificial intelligence (AI) accelerator (e.g., a vector processor, which may include vector arithmetic logic units configured efficiently perform operations common to neural networks, such dot products and softmax), a field programmable gate array (FPGA), an application specific integrated circuit (ASIC), a digital signal processor (DSP), or the like. For example, in some circumstances, aspects of embodiments of the present disclosure are implemented in program instructions that are stored in a non-volatile computer readable memory where, when executed by the electronic circuit (e.g., a CPU, a GPU, an AI accelerator, or combinations thereof), perform the operations described. The operations performed by the classification system 100 may be performed by a single electronic circuit (e.g., a single CPU, a single GPU, or the like) or may be allocated between multiple electronic circuits (e.g., multiple GPUs or a CPU in conjunction with a GPU). The multiple electronic circuits may be local to one another (e.g., located on a same die, located within a same package, or located within a same embedded device or computer system) and/or may be remote from one other (e.g., in communication over a network such as a local personal area network such as Bluetooth®, over a local area network such as a local wired and/or wireless network, and/or over wide area network such as the internet, such a case where some operations are performed locally and other operations are performed on a server hosted by a cloud computing service). One or more electronic circuits operating to implement the classification system 100 may be referred to herein as a computer or a computer system, which may include memory storing instructions that, when executed by the one or more electronic circuits, implement the systems and methods described herein.

It will be understood that, although the terms “first”, “second”, “third”, etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section discussed below could be termed a second element, component, region, layer or section, without departing from the spirit and scope of the inventive concept.

The terminology used herein is for the purpose of describing particular embodiments and is not intended to be limiting of the inventive concept. As used herein, the singular forms “a” and “an” are intended to include the plural forms as well, unless the context clearly indicates otherwise. It will be further understood that the terms “include,” “including,” “comprises,” “comprising,” “has,” “have,” and “having,” when used in this specification, specify the presence of stated features, integers, steps, operations, elements, and/or components, but do not preclude the presence or addition of one or more other features, integers, steps, operations, elements, components, and/or groups thereof.

As used herein, the term “and/or” includes any and all combinations of one or more of the associated listed items. For example, the expression “A and/or B” denotes A, B, or A and B. Expressions such as “one or more of” and “at least one of,” when preceding a list of elements, modify the entire list of elements and do not modify the individual elements of the list. For example, the expression “one or more of A, B, and C,” “at least one of A, B, or C,” “at least one of A, B, and C,” and “at least one selected from the group consisting of A, B, and C” indicates only A, only B, only C, both A and B, both A and C, both B and C, or all of A, B, and C.

Further, the use of “may” when describing embodiments of the inventive concept refers to “one or more embodiments of the inventive concept. ” Also, the term “exemplary”is intended to refer to an example or illustration.

It will be understood that when an element or layer is referred to as being “on”, “connected to”, “coupled to”, or “adjacent” another element or layer, it can be directly on, connected to, coupled to, or adjacent the other element or layer, or one or more intervening elements or layers may be present. When an element or layer is referred to as being “directly on,” “directly connected to”, “directly coupled to”, “in contact with”, “in direct contact with”, or “immediately adjacent” another element or layer, there are no intervening elements or layers present.

As used herein, the term “substantially,” “about,” and similar terms are used as terms of approximation and not as terms of degree, and are intended to account for the inherent variations in measured or calculated values that would be recognized by those of ordinary skill in the art.

As used herein, the terms “use,” “using,” and “used” may be considered synonymous with the terms “utilize,”“utilizing,”and “utilized,”respectively.

When one or more embodiments may be implemented differently, a specific process order may be performed differently from the described order. For example, (i) the disclosed operations of a process are merely examples, and may involve various additional operations not explicitly covered, and (ii) the temporal order of the operations may be varied.

Unless otherwise defined, all terms (including technical and scientific terms) used herein have the same meaning as commonly understood by one of ordinary skill in the art to which the present inventive concept belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Although aspects of some example embodiments of the system and method for biomarker detection have been described and illustrated herein, various modifications and variations may be implemented, as would be understood by a person having ordinary skill in the art, without departing from the spirit and scope of embodiments according to the present disclosure. Accordingly, it is to be understood that a pathology slide manufacturing system and method according to the principles of the present disclosure may be embodiment other than as specifically described herein. The disclosure is also defined in the following claims, and equivalents thereof.

What is claimed is:

1. A method of classifying a tissue sample by a classification system based on machine learning, the method comprising:
identifying, by the classification system, a plurality of tiles corresponding to whole-slide image data of the tissue sample;
generating, by the classification system, a plurality of semantic masks corresponding to the plurality of tiles, each one of the plurality of semantic masks identifying a cell boundary and a cell type of each cell within a corresponding tile of the plurality of tiles;
generating, by the classification system, a plurality of cellular features for each tile of the plurality of tiles based on a corresponding one of the plurality of semantic masks; and
classifying, by the classification system, the tissue sample based on the plurality of cellular features for each one of the plurality of tiles.

2. The method of claim 1, wherein the identifying the plurality of tiles comprises:
receiving, by the classification system, the whole-slide image data corresponding to the tissue sample; and
extracting, by the classification system, the plurality of tiles from the whole-slide image data.

3. The method of claim 1, wherein the whole-slide image data comprises at least one digitized image of the tissue sample of a patient that is stained with hematoxylin and eosin (H&E) dyes or a region-of-interest (ROI) map.

4. The method of claim 1, wherein the identifying the plurality of tiles further comprises:
performing stain normalizing, by the classification system, based on the plurality of tiles to generate a plurality of normalized tiles.

5. The method of claim 4, wherein the performing stain normalizing comprises:
generating, by a first model of the classification system, the plurality of normalized tiles based on the plurality of tiles,
wherein the first model comprises a fully convolutional neural network.

6. The method of claim 1, wherein the generating the semantic masks comprises:
encoding, by an encoder of the classification system, the a tile of the plurality of tiles to generate encoded data corresponding to the tile;
generating, by a segmentation decoder of the classification system, a segmentation mask corresponding to the tile based on the encoded data, the segmentation mask identifying the cell boundary of each cell within the tile; and
classifying, by a classification decoder of the classification system, the cell type of each cell within the segmentation mask as one of a plurality of cell type categories;
generating a semantic mask of the plurality of semantic masks to indicate the cell boundary and the cell type of each cell with the tile.

7. The method of claim 6, wherein the plurality of cell type categories comprises a tumor cell, a lymphocyte cell, and other.

8. The method of claim 1, wherein the generating the plurality of cellular features for each tile of the plurality of tiles comprises:
generating a plurality of nuclear-level features for the tile based on the corresponding one of the plurality of semantic masks;
generating a plurality of tile-level features by aggregating the plurality of nuclear-level features; and
extracting the plurality of cellular features from the plurality of tile-level features.

9. The method of claim 8, wherein the generating the plurality of nuclear-level features for the tile comprises:
computing a plurality of nuclear morphology features for each cell having a tumor cell type within the corresponding one of the plurality of semantic masks,
wherein the plurality of nuclear morphology features comprises at least one of:
basic geometric features comprising shape, size, and circularity of a nucleus of the cell having the tumor cell type;
first-order statistics of gray-level intensity inside the nucleus;
texture features derived from gray-level co-occurrence matrix of the nucleus;
advanced morphology features for characterizing irregularity of the nucleus;
chromatin distribution features of the nucleus;
nuclear boundary signature of the nucleus; or
curvature features of the nucleus, and
wherein the plurality of nuclear-level features comprises a collection of nuclear morphology features of all nuclei of cells having the tumor cell type within the corresponding one of the plurality of semantic masks.

10. The method of claim 8, wherein the generating the plurality of tile-level features comprises:
calculating a statistical mean of ones of the nuclear-level features associated with cells having a tumor cell type within the corresponding one of the plurality of semantic masks to generate a mean vector;
calculating a standard deviation of the ones of the nuclear-level features associated with cells having the tumor cell type to generate a standard deviation vector; and
determining spatial distribution features of cells within the corresponding one of the plurality of semantic masks to generate one or more spatial distribution vectors,
wherein the plurality of tile-level features comprises the mean vector, the standard deviation vector, and the one or more spatial distribution vectors.

11. The method of claim 10, wherein the spatial distribution features comprises:
density of each type of cell of cells within the corresponding one of the plurality of semantic masks; and
average distances between cells within the corresponding one of the plurality of semantic masks.

12. The method of claim 8, wherein the extracting the plurality of cellular features comprises:
removing one or more of the plurality of tile-level features that have low variance across cells or high correlation with other ones of the plurality of tile-level features; and
normalizing remaining ones of the tile-level features to generate the plurality of cellular features.

13. The method of claim 1, wherein the classifying the tissue sample comprises:
generating, by an attention-based aggregator of the classification system, slide-level features based on the plurality of cellular features for each one of the plurality of tiles; and
classifying, by a slide classifier of the classification system, the tissue sample based on the slide-level features.

14. The method of claim 1, wherein the classifying of the tissue sample comprises:
identifying the tissue sample as containing a first subtype of diffuse large B-cell lymphoma (DLBCL) or a second subtype of DLBCL.

15. The method of claim 14, wherein the first subtype comprises a germinal center B-cell-like (GCB) subtype, and
wherein the second subtype comprises an activated B-cell-like (ABC) subtype.

16. A classification system for classifying a tissue sample, the classification system comprising:
a processor; and
a memory storing instructions that, when executed on the processor, cause the processor to perform:
identifying a plurality of tiles corresponding to whole-slide image data of the tissue sample;
generating a plurality of semantic masks corresponding to the plurality of tiles, each one of the plurality of semantic masks identifying a cell boundary and a cell type of each cell within the tile;
generating a plurality of cellular features for each tile of the plurality of tiles based on a corresponding one of the plurality of semantic masks; and
classifying the tissue sample based on the plurality of cellular features for each one of the plurality of tiles.

17. The classification system of claim 16, wherein the generating the semantic masks comprises:
encoding, by an encoder of the classification system, the a tile of the plurality of tiles to generate encoded data corresponding to the tile;
generating, by a segmentation decoder of the classification system, a segmentation mask corresponding to the tile based on the encoded data, the segmentation mask identifying the cell boundary of each cell within the tile; and
classifying, by a classification decoder of the classification system, the cell type of each cell within the segmentation mask as one of a plurality of cell type categories;
generating a semantic mask of the plurality of semantic masks to indicate the cell boundary and the cell type of each cell with the tile, and
wherein the plurality of cell type categories comprises a tumor cell, a lymphocyte cell, and other.

18. The classification system of claim 16, wherein the generating the plurality of cellular features for each tile of the plurality of tiles comprises:
generating a plurality of nuclear-level features for the tile based on the corresponding one of the plurality of semantic masks;
generating a plurality of tile-level features by aggregating the plurality of nuclear-level features; and
extracting the plurality of cellular features from the plurality of tile-level features.

19. The classification system of claim 8, wherein the generating the plurality of tile-level features comprises:
calculating a statistical mean of ones of the nuclear-level features associated with cells having a tumor cell type within the corresponding one of the plurality of semantic masks to generate a mean vector;
calculating a standard deviation of the ones of the nuclear-level features associated with cells having the tumor cell type to generate a standard deviation vector; and
determining spatial distribution features of cells within the corresponding one of the plurality of semantic masks to generate one or more spatial distribution vectors,
wherein the plurality of tile-level features comprises the mean vector, the standard deviation vector, and the one or more spatial distribution vectors.

20. The classification system of claim 16, wherein the classifying the tissue sample comprises:
generating, by an attention-based aggregator of the classification system, slide-level features based on the plurality of cellular features for each one of the plurality of tiles; and
classifying, by a slide classifier of the classification system, the tissue sample based on the slide-level features.
